(19) United States
(12) Patent Application Publication
(10) Pub. No.: US 2024/0425793 A1
(43) Pub. Date: Dec. 26, 2024

(54) PRE-PROCESSING APPARATUS AND PRE-PROCESSING METHOD

(71) Applicant: SHIMADZU CORPORATION, Kyoto-shi, Kyoto (JP)

(72) Inventor: Tomohisa HASUNUMA, Kobe-shi, Hyogo (JP)

(73) Assignee: SHIMADZU CORPORATION, Kyoto-shi, Kyoto (JP)

(21) Appl. No.: 18/688,114

(22) PCT Filed: Mar. 7, 2022
(86) PCT No.: PCT/JP2022/009596

§ 371 Date: Jun. 3, 2024

(51) Int. Cl. C12M 1/26

(52) U.S. Cl. CPC C12M 33/04; C12M 33/14

(57) ABSTRACT

A pre-processing apparatus aspirates a liquid component, as pre-processing, from a culture sample in which the liquid component and a solid component are separated by a solid-liquid interface in a container. The pre-processing apparatus includes: a suction tube for aspirating the liquid component; a pump for providing a negative pressure to the suction tube; and a control unit for moving, in a direction to the solid-liquid interface, the suction tube under the negative pressure within the liquid component.

### TECHNICAL FIELD

The present disclosure relates to a pre-processing apparatus and a pre-processing method.

### BACKGROUND ART

A technology has been known in which an analysis such as metabolome analysis is performed by supplying microorganisms and plant's cells to a liquid chromatograph mass spectrometer. Those cell supplied to the liquid chromatograph mass spectrometer have been cultured in a culture medium in a culture vessel. In this kind of technology, a sampling apparatus for sampling a culture medium containing cells and a pre-processing apparatus for pre-processing the cells contained in the sampled culture medium are used.

In the pre-processing apparatus, for example, a centrifugation mechanism is activated for the container accommodating the culture medium. As a result, the culture medium such as cells are accumulated as a solid at the bottom of the container, for example, and the solid components, which are the culture medium, and the liquid components, which are supernatants, are separated from each other via the solid-liquid interface. The solid components and the liquid components can be separately collected by aspirating the liquid components from the container with a suction tube inserted in the container.

To ensure that no liquid component is left in the container, it is contemplated to aspirate the liquid components while the suction tube is placed close to the solid-liquid interface. In this case, it is important to accurately detect the solid-liquid interface.

PTL 1 discloses a technology of capturing an image of a solid-liquid interface while a container is illuminated with uniform light from outside by surface illumination, in order to accurately detect the position of the solid-liquid interface.

### CITATION LIST

### Patent Literature

PTL 1: WO2020/017411

### SUMMARY OF INVENTION

### Technical Problem

The closer the suction tube for aspirating the liquid component is to the solid-liquid interface, the less the liquid component left in the container. However, the aspiration of the liquid component by the suction tube causes a flow of a liquid. Under the influence of the flow of the liquid, the precipitate solid component may be stirred up and the solid component may be aspirated together with the liquid component by the suction tube. The closer the suction tube for aspirating the liquid component is to the solid-liquid interface, the greater the influence of the flow of the liquid is.

An object of the present disclosure is to keep the aspirate clear of a solid component as much as possible, while allowing an increased amount of liquid components to be aspirated from a culture sample.

### Solution to Problem

A pre-processing apparatus according to the present disclosure aspirates, as a pre-processing, a liquid component from a culture sample being separated into the liquid component and a solid component via a solid-liquid interface within a container. The pre-processing apparatus includes: a suction tube for aspirating the liquid component; a pump for providing a negative pressure to the suction tube; and a control unit for moving in a direction to the solid-liquid interface, the suction tube under the negative pressure within the liquid component.

A pre-processing method according to the present disclosure is a pre-processing method for aspirating a liquid component by a suction tube as pre-processing, from a culture sample separated into the liquid component and a solid component via a solid-liquid interface in a container, the pre-processing method including: providing a negative pressure to the suction tube; and moving the suction tube under the negative pressure in a direction to the solid-liquid interface in the liquid component.

### ADVANTAGEOUS EFFECTS OF INVENTION

According to the present disclosure, the aspirate can be kept clear of a solid component as much as possible, while allowing an increased amount of liquid components to be aspirated from a culture sample.

### BRIEF DESCRIPTION OF DRAWINGS

FIG. 1 is a block diagram showing a schematic configuration of an automated pre-processing system.

FIG. 2 is a diagram showing a schematic configuration of a liquid removal mechanism.

FIG. 3 is a diagram showing a procedure for aspirating supernatants.

FIG. 4 is a timing chart illustrating details of a control for aspirating the supernatants.

FIG. 5 is a flowchart illustrating details of a control for aspirating the supernatants (Embodiment 1).

FIG. 6 is a flowchart illustrating details of a control for aspirating the supernatants (Embodiment 2).

### DESCRIPTION OF EMBODIMENTS

Hereinafter, embodiments according to the present disclosure will be described, with reference to the accompanying drawings. Note that like reference signs are used to refer to like or corresponding parts in the drawings, and the description thereof will not be repeated.

### Embodiment 1

### Schematic Configuration of Automated Pre-Processing System

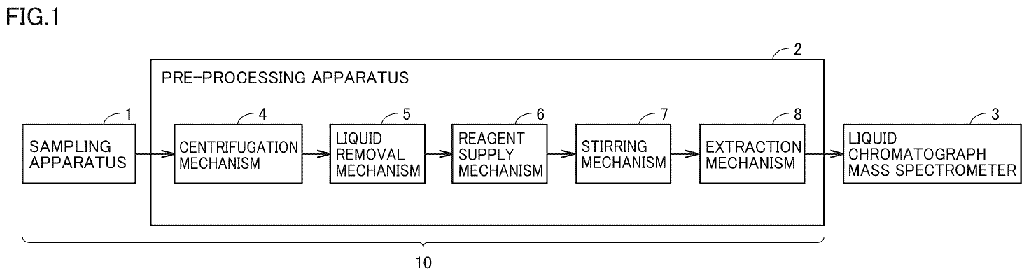

FIG. 1 is a block diagram showing a schematic configuration of an automated pre-processing system 10. The automated pre-processing system 10 is an apparatus for automatically pre-processing an analyte. In the present embodiment, the analyte is, for example, cultured cells, more specifically, bacterial cells.

The automated pre-processing system 10 includes a sampling apparatus 1 and a pre-processing apparatus 2. The cells are subjected to the pre-processing by the automated pre-processing system 10, after which a metabolite of those cells is extracted. The metabolite is supplied to the liquid chromatograph mass spectrometer 3. The liquid chromatograph mass spectrometer 3 is merely one example of an analysis apparatus for analyzing an analyte. Other analysis apparatuses can be used to analyze the analyte.

The sampling apparatus 1 is an apparatus for sampling a liquid from a container (a culture vessel). For example, microbes or plant's cells are cultured within a culture medium in a container called a bioreactor. For example, a stirring member that is rotated by using a magnetic force or an oxygen concentration sensor for sensing the concentration of the dissolved oxygen are provided within the bioreactor. The concentration of the dissolved oxygen is adjusted, while the culture medium being stirred within the bioreactor, and cells are, thereby, cultured within the sampling apparatus 1.

The pre-processing apparatus 2 pre-processes the cells contained in the culture medium (a culture sample) which is sampled from the bioreactor. In the sampling apparatus 1, the culture medium containing the cells is received in a test tube as a container (a sampling container). The pre-processing apparatus 2 includes a centrifugation mechanism 4, a liquid removal mechanism 5, a reagent supply mechanism 6, a stirring mechanism 7, an extraction mechanism 8, etc. These mechanisms sequentially pre-process the cells contained in the culture medium in the test tube.

The centrifugation mechanism 4 provides a centrifugal force to the culture medium in the test tube. This separates the culture medium in the test tube at the solid-liquid interface into a solid component settled at the bottom of the test tube and a liquid component floating over the solid component. The solid component is a culture, for example, cultured cells. The liquid component floating over the solid component is supernatants separated from the culture medium.

The liquid removal mechanism 5 aspirates the supernatants from the test tube. This removes the liquid out of the test tube, leaving the cells in the test tube. The reagent supply mechanism 6 supplies the cells in the test tube with a reagent for extracting a metabolite from the cells. This produces a cell-reagent mixture within the test tube. The stirring mechanism 7 stirs the mixed solution. Stirring the mixed solution allows extraction of the metabolite from the cells, resulting in a suspension.

The extraction mechanism 8 extracts a portion of the suspension as an extract. The extract is supplied to the liquid chromatograph mass spectrometer 3.

### Schematic Configuration of Liquid Removal Mechanism 52

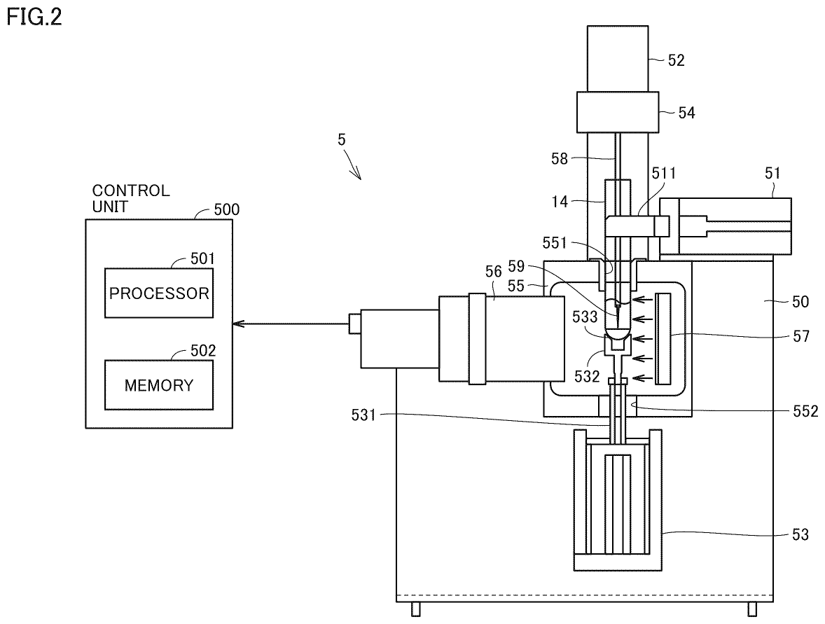

FIG. 2 is a diagram showing a schematic configuration of the liquid removal mechanism 5.

The liquid removal mechanism 5 includes a mounting plate 50 having various members mounted thereon, and a control unit 500. A holding mechanism 51, a drive mechanism 52, a movement mechanism 53, a pump 54, a housing 55, a camera 56, and a surface illumination 57 are mounted on the mounting plate 50.

The holding mechanism 51 includes a pair of displacement portions 511 for holding a test tube 14. The pair of displacement portions 511 are disposed opposing each other in the horizontal direction.

The pump 54 is connected to a nozzle 58. The nozzle 58 has a tip having a tip 59 mounted thereon. The pump 54 provides a negative pressure to the nozzle 58, thereby producing a suction force at the end of the tip 59. A liquid aspirated by the tip 59 passes through a drain (not shown) and collected. The drive mechanism 52 moves the pump 54 and the nozzle 58 in the vertical direction.

The housing 55 is formed in a hollow shape. The test tube 14 is disposed in the housing 55. The housing 55 has a top surface having an opening 551 formed therein for insertion of the test tube 14. The housing 26 has a bottom surface having an opening 552 formed therein for insertion of a shaft 531 of the movement mechanism 53. The surface illumination 57 is disposed within the housing 55. A lens part of the camera 56 and the surface illumination 57 are disposed within the housing 55 on opposite sides of the test tube 14.

The movement mechanism 53 includes the shaft 531 and a pedestal portion 532 at a tip of the shaft 531. The pedestal portion 532 has a recess 533 formed therein. The bottom of the test tube 14 is disposed in the recess 533. The movement mechanism 53 can slide the shaft 531 in the axial direction, thereby adjusting the position of the pedestal portion 532.

The control unit 500 includes a processor 501 and a memory 502. The processor 501 is, typically, a processing unit such as a central processing unit (CPU) or a multi-processing unit (MPU), for example. The processor 501 reads and executes a program stored in the memory 502, thereby implementing various processing of the liquid removal mechanism 5. While FIG. 2 illustrates the control unit 500 as including a single processor 501, it should be noted that the control unit 500 may include multiple processors 501.

The memory 502 is implemented in a non-volatile memory such as a random access memory (RAM), a read only memory (ROM), and a flash memory. Besides the program executed by the processor 501, the memory 502 stores various data such as durations of controls for the drive mechanism 52 and the pump 54.

Note that if the memory 502 is capable of recording programs in a non-transitory fashion in a form readable by the processor 101, the memory 502 may be a compact disc-read only memory (CD-ROM), a digital versatile disk-read only memory (DVD-ROM), a universal serial bus (USB) memory, a memory card, a flexible disk (FD), a hard disk, a solid state drive (SSD), a magnetic tape, a cassette tape, a magnetic optical disc (MO), a MiniDisc (MD), an integrated circuit (IC) cart (except for memory cards), an optical card, a mask ROM, or an EPROM.

The control unit 500 transmits a control signal to the drive mechanism 52, the pump 54, and the camera 56. The drive mechanism 52 moves the nozzle 58 in the vertical direction, based on the control signal. This causes the nozzle 58 to be inserted into the test tube 14. This also controls the position of the tip 59 mounted on the tip of the nozzle 58. The pump 54 provides a negative pressure to the nozzle 58, based on the control signal. This produces a suction force at the nozzle 58. The camera 56 captures an image based on the control signal and transmits the image to the control unit 500. This allows the control unit 500 to obtain an image including the solid-liquid interface of the culture sample in the test tube 14.

### Procedure for Liquid Removal Mechanism 5 to Aspirate Supernatants

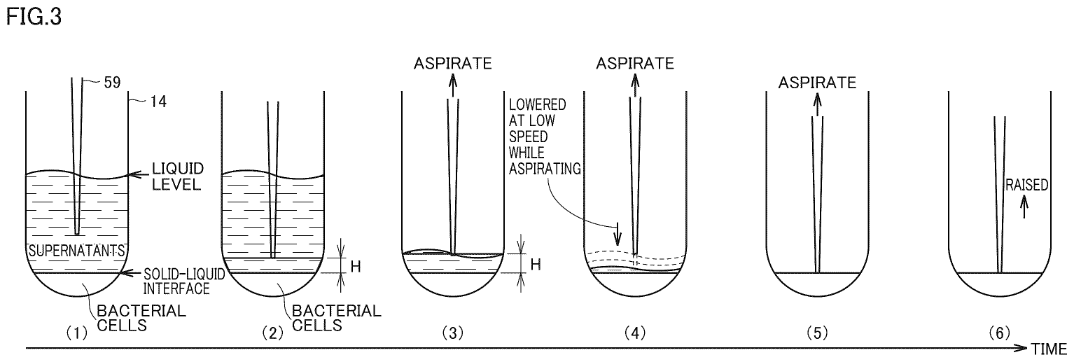

FIG. 3 is a diagram showing a procedure for aspirating supernatants. For example, the liquid removal mechanism 5 aspirates supernatants in the test tube 14, following the procedure described below. The test tube 14 contains supernatants and bacterial cells, being separated by the solid-liquid interface.

Initially, the tip 59 is inserted into the supernatants from above the test tube 14 ((1) of FIG. 3). The tip 59 is mounted on the tip of the nozzle 58 (see FIG. 2).

Next, the end of the tip 59 is moved to a position in the supernatants, the position being a distance H away from the solid-liquid interface in the direction toward the liquid level ((2) of FIG. 3). Next, the tip 59 at that position starts the aspiration of the supernatants. This causes the supernatants to be gradually aspirated through the end of the tip 59, reducing the liquid level. Eventually, the liquid level is brought closer to the end of the tip 59 ((3) of FIG. 3).

The distance H can be set between the liquid level and the solid-liquid interface. The greater the distance H is, the closer the end position of the tip 59 is to the liquid level. When the tip 59 aspirates the supernatants with the end of the tip 59 kept at a position close to the liquid level, more bacterial cells can be prevented from being stirred up under the influence of the flow of the supernatants (liquid) caused by the aspiration, as compared to aspirating the supernatants with the end of the tip 59 kept at a position close to the solid-liquid interface. On the other hand, when the tip 59 aspirates the supernatants with the end of the tip 59 kept at a position close to the liquid level, a reduced amount of supernatants is aspirated with the tip 59 at that position. Accordingly, it is contemplated to design the distance H, taking into an account the influence of the flow of the supernatants caused by the aspiration, and an amount of supernatants to be aspirated.

After the liquid level is brought closer to the end of the tip 59, the tip 59 is lowered toward the solid-liquid interface at a low speed, while the tip 59 keeping aspiration of the supernatants ((4) of FIG. 3). This causes the supernatants present in the area from the solid-liquid interface to the distance H to be gradually aspirated by the tip 59.

The closer the end of the tip 59 is brought to the solid-liquid interface, the greater the influence of the flow of the supernatants caused by the aspiration on the bacterial cells is. However, since the tip 59 is moved at a low speed, while continuing the aspiration, such an influence can be minimized.

As the end of the tip 59, eventually, reaches the solid-liquid interface, the lowering of the tip 59 is stopped. The tip 59 continues the aspiration of the supernatants with the end of the tip 59 having reached the solid-liquid interface ((5) of

FIG. 3). This causes the supernatants near the solid-liquid interface to be aspirated by the tip 59.

As is conventional, if the end of the tip 59 is lowered to the solid-liquid interface while a large amount of supernatants is present and the tip 59 initiates the aspiration of the supernatants, bacterial cells may be stirred up under the influence of the flow of the supernatants caused by the aspiration. In the present embodiment, however, since the tip 59 aspirates the supernatants while the end of the tip 59 is being lowered toward the solid-liquid interface, a small amount of supernatants is left in the test tube 14 when the end of the tip 59 reaches the solid-liquid interface. For example, if the tip 59 is moved in conjunction with the position of the liquid level being lowered by the supernatants being aspirated, an extremely small amount of supernatants is left in the test tube 14 when the tip 59 reaches the solid-liquid interface.

Accordingly, the aspiration time required at the solid-liquid interface can be minimized. This reduces the likelihood of bacterial cells being aspirated together with the supernatants.

Furthermore, in the present embodiment, the tip 59 continues the aspiration of the supernatants even before the end of the tip 59 reaches the solid-liquid interface, rather than initiating the aspiration when the end of the tip 59 reaches the solid-liquid interface. Accordingly, the flow of supernatants is not caused abruptly when the end of the tip 59 reaches the solid-liquid interface. Furthermore, the amount of supernatants, itself, forming the flow is small. Accordingly, the likelihood of bacterial cells being stirred up under the influence of the flow of the supernatants can be reduced. As a result, the likelihood of bacterial cells being aspirated together with the supernatants is reduced.

Once all the supernatants have been aspirated, the tip 59 is raised ((6) of FIG. 3). This is the end of the entire procedure for aspirating the supernatants.

### Timing Chart Illustrating Details of Control for Aspirating Supernatants

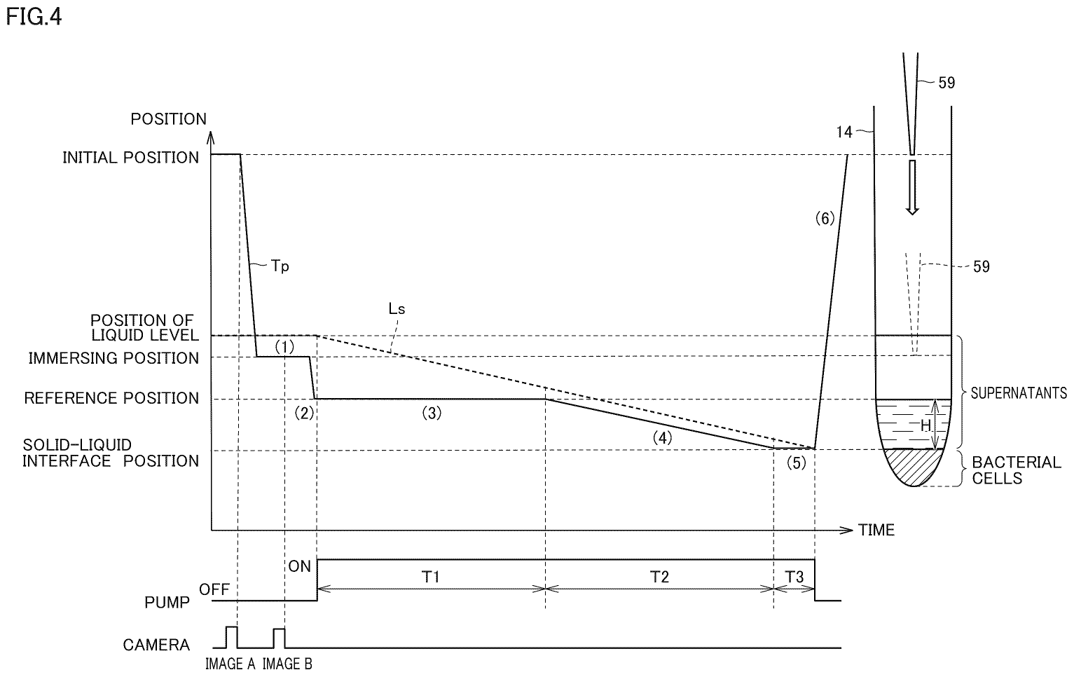

FIG. 4 is a timing chart illustrating details of a control for aspirating the supernatants. Here, the procedure described with reference to FIG. 3 is set forth from the standpoint of controlling the liquid removal mechanism 5.

In FIG. 4, the vertical axis indicates the vertical position of the test tube 14. The horizontal axis shown in FIG. 4 indicates the time axis. What are shown below the time axis of FIG. 4 are on and off timing for the pump 54 and a timing at which the camera 56 captures images of the test tube 14.

In the test tube 14 shown in FIG. 4, bacterial cells and supernatants are separated. The vertical axis indicates the position of the solid-liquid interface between bacterial cells and supernatants, and the position of the liquid level of the supernatants. Parts (1) to (5) of FIG. 4 correspond to (1) to (5) of FIG. 3. In the figure, Tp indicates the end position of the tip 59. Ls indicates the

position of the liquid level changing with the aspiration of the supernatants.

Initially, the end of the tip 59 is at the initial position. At this time, the liquid removal mechanism 5 causes the camera 56 to capture an image A near the solid-liquid interface. Since the end position Tp of the tip 59 is away from the solid-liquid interface, the image A does not include the end of the tip 59.

Next, the liquid removal mechanism 5 moves the end of the tip 59 from the initial position to an immersing position. The immersing position is set lower than the liquid level of the supernatants. The position at which the immersing position is set is also where the end of the tip 59 can be seen in an image of the position of the solid-liquid interface captured at the same imaging position as one for the image A. The liquid removal mechanism 5 cause the camera 56 to capture an image B near the solid-liquid interface while the end of the tip 59 is at the immersing position.

The liquid removal mechanism 5 uses the image A and the image B to detect the solid-liquid interface and the end of the tip 59. For example, the liquid removal mechanism 5 accurately detects the end of the tip 59, based on a difference between the image A and the image B. In other words, if a difference in brightness of each of the pixels is calculated between the image A not including the end of the tip 59 and the image B including the end of the tip 59, a large difference in brightness can be calculated only at the end of the tip 59. Accordingly, based on a result of that calculation, the liquid removal mechanism 5 can accurately detect the end of the tip 59.

Next, the liquid removal mechanism 5 moves the end of the tip 59 from the immersing position to a reference position. The reference position is away from the solid-liquid interface by the distance H in the direction toward the liquid level. The liquid removal mechanism 5 does not turn the pump 54 on when the end of the tip 59 is at the immersing position. The liquid removal mechanism 5 does not turn the pump 54 on while moving the end of the tip 59 from the immersing position to the reference position. However, the liquid removal mechanism 5 may turn the pump 54 on at these timing.

Next, the liquid removal mechanism 5 turns the pump 54 on, while keeping the end of the tip 59 at the reference position. This allows the supernatants to be aspirated through the end of the tip 59. The liquid level Ls falls over time. The liquid removal mechanism 5 lowers the tip 59 at a low speed upon an elapse of time T1 since the pump 54 has been turned on.

As time T1 elapses since the pump 54 has been turned on, the liquid level Ls is closer to the reference position. Note that time T1 may be set so that the tip 59 is lowered after the liquid level Ls is coincide with the reference position. Stated differently, time T1 may be set after the tip 59 has finished the aspiration of the supernatants present above the reference position. Setting time T1 as such allows a larger amount of supernatants to be aspirated at the reference position away from the position of the solid-liquid interface. Note that the control unit 500 may detect changes in pressure at the end of the tip 59 to determine whether the end of the tip 59 is within the supernatants or in contact with the liquid level.

The liquid removal mechanism 5 lowers the end of the tip 59 at a low speed over time T2, from the reference position to the solid-liquid interface. During this time period, the liquid removal mechanism 5 keeps the pump 54 turned on. This allows the supernatants to be gradually aspirated by the tip 59, and the liquid level Ls falls. FIG. 4 shows an example in which the end position Tp of the tip 59 is lowered to be located slightly below the liquid level Ls.

The end of the tip 59 being lowered while being close to the liquid level Ls allows the tip 59 to aspirate the supernatants, constantly away from the position of the solid-liquid interface. This can prevent bacterial cells from being stirred up under the influence of the flow of supernatants caused by the aspiration.

The relationship between the liquid level Ls and the end position Tp of the tip 59 shown in FIG. 4 is by way of example. The lowering rate of the tip 59 may be controlled at a sufficiently slow speed so that the end position Tp of the tip 59 can be lowered along the liquid level Ls.

In particular, if the lowering rate of the tip 59 is sufficiently low, the falling rate of the liquid level Ls due to the aspiration of the supernatants is faster than the lowering rate of the end position Tp of the tip 59. As the tip 59 continues the aspiration of supernatants in such conditions, the position of the liquid level Ls will eventually catch up with the end position Tp of the tip 59. From then on, the aspiration continues while the liquid level Ls and the end position Tp of the tip 59 are coincide with each other. As a result, the tip 59 is lowered at a low speed, while aspirating the supernatants constantly at the liquid level Ls, which is farthest away from the solid-liquid interface. This can further prevent bacterial cells from being stirred up under the influence of the flow of the supernatants caused by the aspiration.

According to the present embodiment, the simple control of slowing the lowering rate of the tip 59 enables the tip 59 to aspirate the supernatants at the liquid level Ls farthest away from the solid-liquid interface. Owing to this, according to the present embodiment, no control is required for determining the relationship between the liquid level Ls and the end position Tp of the tip 59 to allow the tip 59 to continue the aspiration of the supernatants at the position of the liquid level Ls farthest away from the solid-liquid interface.

The liquid removal mechanism 5 stops lowering the tip 59 as time T2 elapses since the start of lowering of the tip 59. At this time, the end position Tp of the tip 59 reaches the solid-liquid interface. The liquid removal mechanism 5 causes the tip 59 to continue the aspiration of the supernatants over time T3, with the end position Tp of the tip 59 having reached the solid-liquid interface. This allows the tip 59 to aspirate a slight amount of supernatants remaining near the solid-liquid interface. The liquid removal mechanism 5 pulls out the tip 59 once time T3 has elapsed. All the supernatants separated above the solid-liquid interface are aspirated with the above control.

In the timing chart of FIG. 4, when the pump 54 is on, the end position Tp of the tip 59 and the liquid level Ls are displaced, keeping an equal distance therebetween. However, the distance between the two may not necessarily be an equal distance. Moreover, the magnitudes of the negative pressure of the pump 54 at time T1, time T2, and time T3 may all be the same or different.

In the present embodiment, the example in which the tip 59 is mounted on the nozzle 58 is described as one aspect of the suction tube. However, a suction tube that is not separated into the nozzle 58 and the tip 59 may be applied to the present embodiment.

In the present embodiment, the end of the tip 59 is moved to the reference position, the pump 54 is turned on, and the pump 54 is, then, kept turned on until the tip 59 finishes the aspiration of the supernatants at the solid-liquid interface. However, the pump 54 may be, temporarily, turned off immediately before the tip 59 is lowered from the reference position to the position of the solid-liquid interface, and the pump 54 may be turned on at the start of the tip 59 being lowered from the reference position to the position of the solid-liquid interface. Moreover, the pump 54 may be turned off upon the completion of the movement of the tip 59 from the reference position to the position of the solid-liquid interface, after which the pump 54 may be turned on.

In the present embodiment, the end of the tip 59 is moved from the initial position to the immersing position, and the end of the tip 59 is, then, moved from the immersing position to the reference position. However, the initial position may be set to the immersing position. Alternatively, the end of the tip 59 may be moved to the reference position directly from the initial position.

### Flowchart Illustrating Details of Control for Aspirating Supernatants

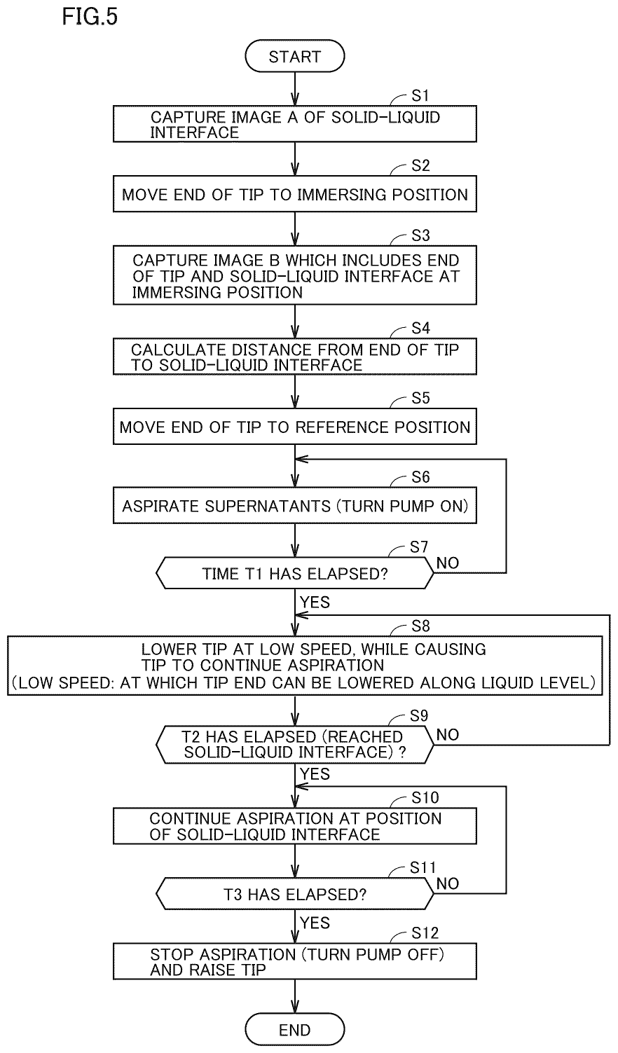

FIG. 5 is a flowchart illustrating details of the control for aspirating the supernatants. The control for the processing based on the flowchart is performed by the control unit 500 included in the liquid removal mechanism 5.

The control unit 500, initially, captures the image A of the solid-liquid interface (step S1). At this time, the end of the tip 59 is at the initial position, the image A therefore does not include the tip 59. Next, the control unit 500 moves the end of the tip 59 from the initial position to the immersing position (step S2). More specifically, the control unit 500 causes the drive mechanism 52 to move the tip 59 to the immersing position. This gets the end of the tip 59 within the supernatants.

Next, the control unit 500 captures the image B which includes the end of the tip and the solid-liquid interface at the immersing position (step S3). Next, using the image A and the image B, the control unit 500 determines the position of the solid-liquid interface and the end position of the tip 59 to calculate the distance from the end of the tip 59 to the solid-liquid interface (step S4).

Next, the control unit 500 moves the end of the tip 59 to the reference position (step S5). The reference position is set to a position the distance H upward from the solid-liquid interface. The control unit 500 subtracts H from the distance calculated in step S4 to calculate a required distance of movement to guide the end of the tip 59 to the reference position. Based on the calculated distance of movement, the control unit 500 lowers the tip 59, thereby moving the end of the tip 59 to the reference position.

Next, the control unit 500 causes the tip 59 to aspirate the supernatants, while keeping the end of the tip 59 at the reference position (step S6). More specifically, the control unit 500 drives the pump 54, while keeping the end of the tip 59 at the reference position. This provides a negative pressure within the tip 59, allowing the supernatants to be aspirated by the tip 59.

Next, the control unit 500 determines whether time T1 has elapsed (step S7). The data at time T1 is pre-stored in the memory 502 of the control unit 500. Time T1 is, for example, a time required for the liquid level to reach a reference surface or be lowered to near the liquid level since the start of the aspiration by the tip 59 at the reference position. Time T1 has been adjusted at the design phase.

If time T1 has not elapsed, the control unit 500 returns to step S6 and causes the tip 59 to continue the aspiration of the supernatants. If time T1 has elapsed, the control unit 500 lowers the tip 59 at a low speed, while causing the tip 59 to continue the aspiration of the supernatants (step S8). The lowering rate of the tip 59 is, for example, a sufficiently slow speed at which the end of the tip 59 can be lowered along the liquid level that is falling due to the supernatants being aspirated by the tip 59.

The control unit 500 also limits the lowering rate of the tip 59 to achieve such a speed. This allows the end of the tip 59 to move from the reference position toward the solid-liquid interface, while being kept near the liquid level. Note that the control unit 500 may lower the tip 59 at a sufficiently slow speed at which the end of the tip 59 can be lowered along the liquid level falling due to the supernatants being aspirated by the tip 59, or lower the tip 59 at a little faster speed than such a speed.

Next, the control unit 500 determines whether time T2 has elapsed (step S9). The data at time T2 is pre-stored in the memory 502 of the control unit 500. For example, time T2 is a time required for the tip 59 at the reference position to reach the solid-liquid interface. Time T2 has been adjusted at the design phase.

If time T2 has not elapsed, the control unit 500 returns to step S8 and continues the control of lowering the tip 59 while causing the tip 59 to aspirate the supernatants. If time T2 has elapsed, the control unit 500 stops lowering the tip 59 and causes the tip 59 to continue to aspirate the supernatants at the position of the solid-liquid interface (step S10).

Next, the control unit 500 determines whether time T3 has elapsed (step S11). The data at time T3 is pre-stored in the memory 502 of the control unit 500. Time T3 is, for example, a time required for the tip 59, having reached the solid-liquid interface, to aspirate the supernatants that are remaining above the solid-liquid interface. Time T3 has been adjusted at the design phase.

If time T3 has not elapsed, the control unit 500 returns to step S10 and continues the control of causing the tip 59 to aspirate the supernatants at the position of the solid-liquid interface. If time T3 has elapsed, the control unit 500 causes the tip 59 to stop the aspiration of the supernatants, and raises the tip 59 (step S12). More specifically, the control unit 500 stops driving of the pump 54, and causes the drive mechanism 52 to move the tip 59 to the initial position. The control unit 500 thereby ends the processing based on the flowchart.

Experiment employing an E. coli culture medium was performed in accordance with the procedure described as the present embodiment. As a result, it was confirmed that the supernatants can be aspirated with an accuracy of the remaining supernatants being 100 uL or less. The present embodiment is applicable to, for example, the automation of an apparatus such as an aspirator for removing a liquid of an upper layer while leaving the precipitate.

### Embodiment 2

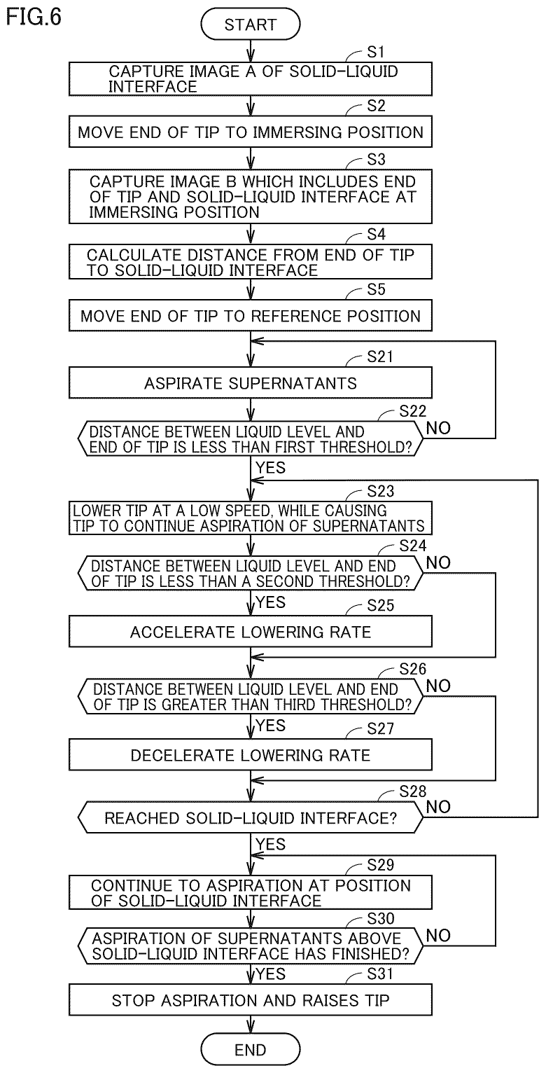

Next, Embodiment 2 is described. FIG. 6 is a flowchart illustrating details of a control for aspirating supernatants. A processing based on the flowchart is performed by a control unit 500 included in a liquid removal mechanism 5.

In Embodiment 1, the control unit 500 controls the movement of the tip 59 and the driving of the pump 54, based on times T1, T2, and T3 set at the design phase. In Embodiment 2, the control unit 500 controls the movement of a tip 59 and the driving of a pump 54, based on the position of the tip 59 and the position of the liquid level. Embodiment 2 is the same as Embodiment 1, except for this respect. Note that, in Embodiment 2, the position of the tip 59 and the position of the liquid level are detected based on an image capture by a camera 56, for example.

Referring to the flowchart of FIG. 6, Embodiment 2 is described. The control unit 500, initially, performs steps S1, S2, S3, S4, and S5. These steps are the same as steps S1 through S5 of FIG. 5. Accordingly, the description thereof will not be repeated here.

The process of step S5 moves the end of the tip 59 to a reference position. The control unit 500 causes the tip 59 to aspirate the supernatants, while keeping the end of the tip 59 at the reference position (step S21). More specifically, the control unit 500 drives the pump 54, while keeping the end of the tip 59 at the reference position. This provides a negative pressure within the tip 59, allowing the supernatants to be aspirated by the tip 59.

Next, the control unit 500 determines whether the distance between the liquid level and the end of the tip 59 is less than a first threshold (step S22). As the aspiration of the supernatants progresses at the reference position, the liquid level falls, bringing the position of the liquid level closer to the end position of the tip 59. In Embodiment 2, the control unit 500 lowers the tip 59 if the distance between the liquid level and the end of the tip 59 is less than the first threshold. The first threshold is pre-stored in a memory 502 of the control unit 500. The control unit 500 may be configured to set the first threshold to an arbitrary value.

If the distance between the liquid level and the end of the tip 59 is not less than the first threshold, the control unit 500 returns step S21 and causes the tip 59 to continue the aspiration of the supernatants. If the distance between the liquid level and the end of the tip 59 is less than the first threshold, the control unit 500 lowers the tip 59 at a low speed, while causing the tip 59 to continue the aspiration of the supernatants (step S23). This moves the end of the tip 59 at the reference position toward the solid-liquid interface.

The control unit 500, while lowering the tip 59, determines whether the distance between the liquid level and the end of the tip 59 is less than a second threshold (step S24). As the control unit 500 causes the tip 59 to aspirate the supernatants while lowering the tip 59, the end position of the tip 59 is lowered, while the position of the liquid level is falling. In Embodiment 2, the control unit 500 lowers the tip 59 while limiting the distance from the end of the tip 59 to the liquid level to a range from the second threshold to a third threshold (provided that the second threshold is less than the third threshold).

Accordingly, if the distance between the liquid level and the end of the tip 59 is less than the second threshold, the control unit 500 accelerates the lowering rate of the tip 59 (step S25). If the distance between the liquid level and the end of the tip 59 is greater than the third threshold (YES in step S26), in contrast, the control unit 500 decelerates the lowering rate of the tip 59 (step S27).

For example, if lowering of the tip 59 is desired, while keeping the end position of the tip 59 near the liquid level, it is contemplated to set the third threshold to a smaller value. Note that the control unit 500 may detect changes in pressure at the end of the tip 59 to determine whether the end of the tip 59 is within the supernatants or in contact with the liquid level.

The second threshold and the third threshold are pre-stored in the memory 502 of the control unit 500. The control unit 500 may be configured to set the second threshold and the third threshold to arbitrary values.

Next, the control unit 500 determines whether the end of the tip 59 has reached the solid-liquid interface (step S28). If the end of the tip 59 has not reached the solid-liquid interface, the control unit 500 continues the process of step S23. If the end of the tip 59 has reached the solid-liquid interface, the control unit 500 stops lowering the tip 59, and causes the tip 59 to continue to aspirate the supernatants at the position of the solid-liquid interface (step S29).

Next, the control unit 500 determines whether the aspiration of the supernatants remaining above the solid-liquid interface has finished (step S30). Specifically, the control unit 500 determines whether the liquid level has reached the solid-liquid interface and thereby disappeared. If the tip 59 has not finished the aspiration of the supernatants remaining above the solid-liquid interface yet, the control unit 500 causes the tip 59 to continue to aspirate the supernatants (step S29). If the tip 59 has finished the aspiration of the supernatants remaining above the solid-liquid interface, the control unit 500 causes the tip 59 to stop the aspiration of the supernatants and raises the tip 59 (step S31). The control unit 500 thereby ends the processing based on the flowchart.

In Embodiments 1 and 2 described above, the procedures for the aspiration of supernatants have been described, intended to remove, from a culture sample separated into bacterial cells and supernatants, the supernatants and collect the bacterial cells. However, the present disclosure is, of course, applicable to the aspiration of supernatants intended to remove, from a culture sample separated into bacterial cells and supernatants, the bacterial cells and collect the supernatants. In this case, the position at which the supernatants are aspirated at time T3 of FIG. 4 may be set slightly above the solid-liquid interface. This can further keep the supernatants to be collected clear of bacterial cells.

### Aspects

It will be appreciated by a person skilled in the art that the embodiments described above and variations thereof are specific examples of the following aspects.

(Item 1) A pre-processing apparatus according to one aspect aspirates, as a pre-processing, a liquid component from a culture sample being separated into the liquid component and a solid component via a solid-liquid interface within a container. The pre-processing apparatus includes: a suction tube for aspirating the liquid component; a pump for providing a negative pressure to the suction tube; and a control unit for moving in a direction to the solid-liquid interface, the suction tube under the negative pressure within the liquid component.

According to the pre-processing apparatus of Item 1, the aspirate can be kept clear of a solid component as much as possible, while allowing an increased amount of liquid components to be aspirated from a culture sample.

(Item 2) In the pre-processing apparatus described in Item 1, the control unit holds the suction tube under the negative pressure in the liquid component at a first position a first distance away from the solid-liquid interface in a direction toward a liquid level of the culture sample, and moves the suction tube under the negative pressure in a direction to the solid-liquid interface in the liquid component in response to a predetermined condition for moving the suction tube being met.

According to the pre-processing apparatus of Item 2, at least some of the liquid component present from the liquid level to the first position can be aspirated prior to the movement of the suction tube under the negative pressure in the direction to the solid-liquid interface in the liquid component.

(Item 3) In the pre-processing apparatus described in Item 2, the control unit moves the suction tube under the negative pressure from the first position to a second position in a direction to the solid-liquid interface, in response to the predetermined condition being met, and holds the suction tube under the negative pressure at the second position.

According to the pre-processing apparatus of Item 3, the liquid component present from the liquid level to the first position can be aspirated until the predetermined condition is met. The liquid component can also be aspirate at the second position closer to the solid-liquid interface than the first position.

(Item 4) In the pre-processing apparatus described in Item 3, the predetermined condition includes that a set time has elapsed.

According to the pre-processing apparatus of Item 4, the liquid component present from the liquid level to the first position can be aspirated until the elapse of the set time.

(Item 5) In the pre-processing apparatus described in Item 3 or 4, the second position is the solid-liquid interface.

According to the pre-processing apparatus of Item 5, the liquid component remaining near the solid-liquid interface can be aspirated.

(Item 6) In the pre-processing apparatus described any one of Items 3 to 5, the control unit maintains the suction tube under the negative pressure from when the negative pressure is provided to the suction tube within the liquid component until the control unit holds the suction tube at the second position.

According to the pre-processing apparatus of Item 6, the liquid component can be efficiently aspirated. Moreover, since the suction tube being under the negative pressure continues, the flow of the liquid component caused by the aspiration can be stabilized, as compared to a control in which the negative pressure is turned off part way through the aspiration and turned back on. As a result, the solid component can be prevented from being stirred up under the influence of the flow of the liquid component caused by the aspiration.

(Item 7) In the pre-processing apparatus described in any one of Items 1 to 6, the control unit limits a speed of movement of the suction tube so that a portion of the suction tube for aspirating the liquid component is along a liquid level of the liquid component that is moved by the liquid component being aspirated by the suction tube.

According to the pre-processing apparatus of Item 7, limiting the speed of movement of the suction tube enable the aspiration of the liquid component to continue constantly at the position of the liquid level farthest away from the solid-liquid interface. As a result, the simple control of limiting the speed of movement can efficiently prevent the solid component from being stirred up under the influence of the flow of the liquid component caused by the aspiration.

(Item 8) In the pre-processing apparatus described in any one of Items 1 to 6, when moving the suction tube under the negative pressure in the liquid component in the direction to the solid-liquid interface, the control unit controls a distance between the liquid level of the liquid component and a position at which the suction tube aspirates the liquid component.

According to the pre-processing apparatus of Item 8, the aspiration can be continued, while keeping the position at which the liquid component is aspirated by the suction tube lower than the liquid level as much as possible. As a result, the liquid component can be efficiently aspirated.

(Item 9) In a pre-processing method described in Item 9, the pre-processing method is for aspirating a liquid component by a suction tube as pre-processing, from a culture sample separated into the liquid component and a solid component via a solid-liquid interface in a container, the pre-processing method including: providing a negative pressure to the suction tube; and moving the suction tube under the negative pressure in a direction to the solid-liquid interface in the liquid component.

According to the pre-processing method of Item 9, the aspirate can be kept clear of a solid component as much as possible, while allowing an increased amount of liquid components to be aspirated from a culture sample.

The presently disclosed embodiments above should be considered illustrative in all aspects and do not limit the present disclosure. The scope of the present invention is defined by the appended claims, rather than by the description of the embodiment set forth above. All changes which come within the meaning and range of equivalency of the appended claims are to be embraced within their scope.

### REFERENCE SIGNS LIST

1 sampling apparatus; 2 pre-processing apparatus; 3 liquid chromatograph mass spectrometer; 4 centrifugation mechanism; 5 liquid removal mechanism; 6 reagent supply mechanism; 7 stirring mechanism; 8 extraction mechanism; 10 automated pre-processing system; 14 test tube; 50 mounting plate; 51 holding mechanism; 52 drive mechanism; 53 movement mechanism; 54 pump; 55 housing; 56 camera; 57 surface illumination; 58 nozzle; 59 tip; 500 control unit; 501 processor; 502 memory; 511 displacement portion; 531 shaft; 532 pedestal portion; 533 recess; 551, 552 opening; Ls position of liquid level; and Tp tip end position.

1. A pre-processing apparatus for aspirating, as a pre-processing, a liquid component from a culture sample being separated into the liquid component and a solid component via a solid-liquid interface within a container, the pre-processing apparatus, comprising:
a suction tube for aspirating the liquid component;
a pump for providing a negative pressure to the suction tube; and
a control unit for moving in a direction to the solid-liquid interface, the suction tube under the negative pressure within the liquid component.

2. The pre-processing apparatus according to claim 1, wherein the control unit holds the suction tube under the negative pressure in the liquid component at a first position a first distance away from the solid-liquid interface in a direction toward a liquid level of the culture sample, and moves the suction tube under the negative pressure in a direction to the solid-liquid interface in the liquid component in response to a predetermined condition for moving the suction tube being met.

3. The pre-processing apparatus according to claim 2, wherein the control unit moves the suction tube under the negative pressure from the first position to a second position in a direction to the solid-liquid interface, in response to the predetermined condition being met, and holds the suction tube under the negative pressure at the second position.

4. The pre-processing apparatus according to claim 3, wherein the predetermined condition includes that a set time has elapsed.

5. The pre-processing apparatus according to claim 3, wherein
the second position is the solid-liquid interface.

6. The pre-processing apparatus according to claim 3, wherein
the control unit maintains the suction tube under the negative pressure from when the negative pressure is provided to the suction tube within the liquid component until the control unit holds the suction tube at the second position.

7. The pre-processing apparatus according to claim 1, wherein
the control unit limits a speed of movement of the suction tube so that a portion of the suction tube for aspirating the liquid component is along a liquid level of the liquid component that is moved by the liquid component being aspirated by the suction tube.

8. The pre-processing apparatus according to claim 1, wherein
when moving the suction tube under the negative pressure in the liquid component in the direction to the solid-liquid interface, the control unit controls a distance between the liquid level of the liquid component and a position at which the suction tube aspirates the liquid component.

9. A pre-processing method for aspirating a liquid component by a suction tube as pre-processing, from a culture sample separated into the liquid component and a solid component via a solid-liquid interface in a container, the pre-processing method comprising:
providing a negative pressure to the suction tube; and
moving the suction tube under the negative pressure in a direction to the solid-liquid interface in the liquid component.
